(19) United States
(12) Patent Application Publication
(10) Pub. No.: US 2025/0191748 A1
(43) Pub. Date: Jun. 12, 2025

(54) SYSTEM AND METHOD FOR MANAGING HEALTH IN AN AUTOMOBILE USING ARTIFICIAL INTELLIGENCE TECHNIQUES

(71) Applicant: Robert Hanson, II, Mobile, AL (US)

(72) Inventor: Robert Hanson, II, Mobile, AL (US)

(21) Appl. No.: 18/974,768

(22) Filed: Dec. 9, 2024

Related U.S. Application Data
(60) Provisional application No. 63/608,184, filed on Dec. 8, 2023.

(51) Int. Cl. G16H 40/63

(52) U.S. Cl. CPC G16H 40/63

(57) ABSTRACT

A system and method for managing health in an automobile is provided. Generally, the system is designed to identify users of the system and monitor health of the users via medical devices and artificial intelligence techniques. The system generally comprises a computing device having a user interface, medical device having at least one sensor configured to measure patient data of a user, display operably connected to said computing device and said medical device, processor operably connected to said computing device, medical device, and display, and non-transitory computer-readable medium coupled to said processor and having instructions stored thereon.

### CROSS REFERENCES

This application claims priority to U.S. Provisional Application Ser. No. 63/608,184, filed on Dec. 8, 2023, in which application is incorporated herein in its entirety by reference.

### FIELD OF THE DISCLOSURE

The subject matter of the present disclosure refers generally to a system and method for managing health in an automobile via a computing device, medical device, display, and machine learning technique.

### BACKGROUND

Artificial intelligence has become increasingly integrated into modern vehicles, transforming the driving experience through advanced driver assistance systems and autonomous capabilities. These AI systems typically focus on external factors such as road conditions, traffic patterns, and obstacle detection to enhance safety and navigation. However, the internal monitoring of driver health and wellness has historically received less attention in automotive AI applications. Medical emergencies while driving present significant safety risks to both the affected driver and others on the road. Traditional vehicle systems lack the capability to detect and respond to medical situations such as heart attacks, diabetic episodes, or other acute health events that may occur during transit. Furthermore, existing health monitoring devices are typically standalone units that do not integrate with vehicle systems or provide contextual information relevant to driving conditions.

The incorporation of displays in vehicles has evolved from simple dashboard gauges to sophisticated digital interfaces. These displays traditionally present information related to vehicle operation, entertainment, and navigation. While some vehicles include basic driver monitoring systems focused on drowsiness detection through steering patterns or eye tracking, comprehensive health monitoring capabilities have been limited. Current automotive displays typically present information in fixed layouts that may not adequately adapt to different medical monitoring needs or emergency situations. Additionally, existing systems often lack the capability to securely manage and share health information between vehicles and healthcare providers, limiting the potential for real-time medical assistance during emergencies.

The integration of artificial intelligence in health monitoring applications has shown promise in various contexts, but its application in automotive environments presents unique challenges. These challenges include the need for real-time processing of health data, secure management of medical information, and appropriate presentation of health alerts without causing driver distraction. While these challenges have slowed development, the increasing diagnoses of chronic conditions worldwide have only sharpened the need for this class of technology.

Accordingly, there is a need in the art for a system and method for the real-time monitoring of a user's health while in a vehicle.

### SUMMARY

A system and method for managing health in a vehicle is provided. In one aspect, the present invention is a system for managing and displaying health related content on a display within a vehicle. In another aspect, the present invention is a system for linking and controlling a plurality of devices, some of which are computing devices, some of which are displays, and some of which are medical devices comprising sensors. In yet another aspect, the present invention is a method of applying machine learning techniques to improve the performance of a system of linked displays, computing devices, and medical devices in a vehicle. In still another aspect, the present invention is a method of utilizing the aforementioned system to enhance health care in the home. Generally, the present invention is a system and method of managing and monitoring health in a vehicle by linking computing devices with one or more medical devices and vehicle displays and applying machine learning techniques to data collected by said system.

The system generally comprises a computing device, medical device, display within a vehicle, processor, medical device, display, and non-transitory computer-readable medium (CRM) coupled to said processor and having instructions stored thereon. Some preferred embodiments further comprise a control board configured to control a display user interface presented on the display. The computing device has a user interface. The medical device comprises at least one sensor configured to measure patient data of a user. The display is operably connected to said computing device and said medical device. The processor is operably connected to the computing device and display. Some preferred embodiments of the system may further comprise a camera operably connected to computing devices, displays, and/or secondary security devices.

A method for displaying and managing content on a display within a vehicle is furthermore provided. This method encompasses the steps and processes described and depicted in the present disclosure. The method may involve a series of operations designed to optimize content presentation on various types of display devices. It may include procedures for content preparation, formatting, and rendering that are specifically tailored to enhance visibility, readability, and user engagement. The method may also incorporate adaptive techniques to adjust content display based on factors such as device characteristics, user preferences, and environmental conditions. The content management method may involve sophisticated techniques for content curation, scheduling, and delivery. It may include steps for analyzing content relevance, optimizing display timing, and managing content transitions. The method may also incorporate feedback mechanisms to continuously improve content selection and presentation based on user interactions and engagement metrics.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 illustrates a system embodying features consistent with the principles of the present disclosure.

FIG. 2 illustrates a system embodying features consistent with the principles of the present disclosure.

FIG. 3 illustrates a system embodying features consistent with the principles of the present disclosure.

FIG. 4 illustrates a system embodying features consistent with the principles of the present disclosure.

FIG. 5 illustrates a system embodying features consistent with the principles of the present disclosure.

FIG. 6 illustrates a system embodying features consistent with the principles of the present disclosure.

FIG. 7 illustrates a system embodying features consistent with the principles of the present disclosure.

FIG. 8 illustrates the manner in which individual access to data may be granted or limited based on user roles and administrator roles.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For instance, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises”, and grammatical equivalents thereof are used herein to mean that other components, steps, etc. are optionally present. For instance, a system “comprising” components A, B, and C can contain only components A, B, and C, or can contain not only components A, B, and C, but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). As will be evident from the disclosure provided below, the present invention satisfies the need for a system and method capable of monitoring and managing the health of its users.

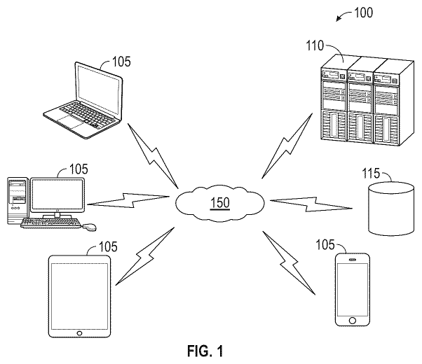

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a streaming device, a “smart” television, a personal digital assistant (PDA), a laptop, a smart phone, a tablet computer, or another type of computation or communication interface 280. Servers 110 may include devices that access, fetch, aggregate, process, search, provide, and/or maintain documents. Although FIG. 1 depicts a preferred embodiment of an environment 100 for the system 400, in other implementations, the environment 100 may contain fewer components, different components, differently arranged components, and/or additional components than those depicted in FIG. 1. Alternatively, or additionally, one or more components of the environment 100 may perform one or more other tasks described as being performed by one or more other components of the environment 100.

As depicted in FIG. 1, one embodiment of the system 400 may comprise a server 110. Although shown as a single server 110 in FIG. 1, a server 110 may, in some implementations, be implemented as multiple devices interlinked together via the network 150, wherein the devices may be distributed over a large geographic area and performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server 110. Additionally, it is understood that multiple servers 110 may be used to operably connect the processor 220 to the database 115 and/or other content repositories. The processor 220 may be operably connected to the server 110 via wired or wireless connection. Types of servers 110 that may be used by the system 400 include, but are not limited to, search servers, document indexing servers, and web servers, or any combination thereof.

Search servers may include one or more computing entities 200 designed to implement a search engine, such as a documents/records search engine, general webpage search engine, etc. Search servers may, for instance, include one or more web servers designed to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, wherein a provided webpage may include a reference to a web server at which the desired information and/or links are located. The references to the web server at which the desired information is located may be included in a frame and/or text box, or as a link to the desired information/document. Document indexing servers may include one or more devices designed to index documents available through networks 150. Document indexing servers may access other servers 110, such as web servers that host content, to index the content. In some implementations, document indexing servers may index documents/records stored by other servers 110 connected to the network 150. Document indexing servers may, for instance, store and index content, information, and documents relating to user accounts and user-generated content. Web servers may include servers 110 that provide webpages to clients 105. For instance, the webpages may be HTML-based webpages. A web server may host one or more websites. As used herein, a website may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database 115 and DBMS.

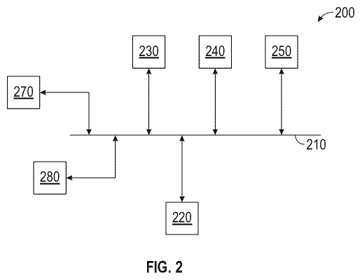

FIG. 2 is an exemplary diagram of a client 105, server 110, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280 (such as wired or wireless communication device). The bus 210 may be defined as one or more conductors that permit communication among the components of the computing entity 200. The processor 220 may be defined as logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

The bus 210 may comprise a high-speed interface 308 and/or a low-speed interface 312 that connects the various components together in a way such they may communicate with one another. A high-speed interface 308 manages bandwidth-intensive operations for computing device 300, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, 511, 711, applications run by a computing entity 200, and wireless communication by a communication interface 280 of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, smart televisions, streaming devices, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, smart televisions, streaming devices, or any other similar device.

Memory 304 stores information within the computing device 300. In some preferred embodiments, memory 304 may include one or more volatile memory units. In another preferred embodiment, memory 304 may include one or more non-volatile memory units. Memory 304 may also include another form of computer-readable medium, such as a magnetic, solid state, or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a controller, scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a streaming device may transfer data to a smartphone, wherein the smartphone may use that data in a manner separate from the streaming device.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

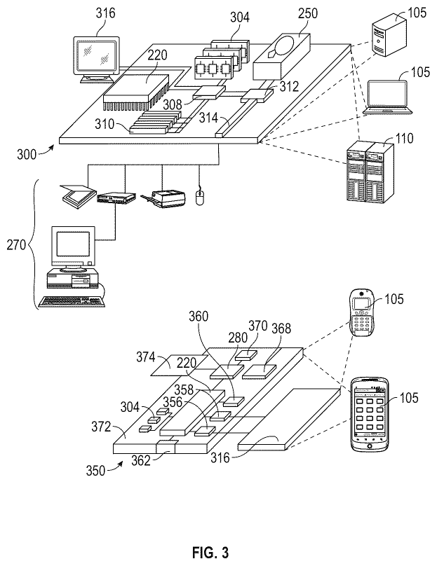

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases 115, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer-readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For instance, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, 511, 711, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), holographic displays, augmented reality displays, virtual reality displays, or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System (EMS), Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0), and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation- and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The system 400 may comprise a power supply, which may be any source of power that provides the system 400 with the required energy. In a preferred embodiment, the power supply may be a stationary power source that has been installed in a way such that it is fastened in place, such as a 3-prong wall outlet. In a preferred embodiment, the stationary power source is connected to the wiring system of a premises, such as a house or a building. In another preferred embodiment, the power supply may be a mobile power source, such as a battery pack, gas-powered generator, alternator, and fuel cell. In a preferred embodiment, the mobile power source does not need to be connected to the wiring system of a premises to provide power to the system but may be capable of connecting to the wiring system of said premises to provide power to a system connected thereto. In another preferred embodiment, the system 400 may comprise multiple power supplies configured to supply power to the system 400 in different circumstances. For instance, the system 400 may be directly plugged into a stationary power source, which may provide power to the system 400 so long as the system does not move out of range of said stationary power source, as well as connected to a mobile power source, which may provide power to the system 400 when the system 400 is not connected to a stationary power source or in situations where the stationary power source ceases to provide power to the system 400. In yet another preferred embodiment, a plurality of solar charging panels may be operably connected to a battery of the system, which may then supply power to the system either directly or via the wiring of the premises. As such, the system 400 may be configured to receive power in a variety of ways without departing from the inventive subject matter described herein.

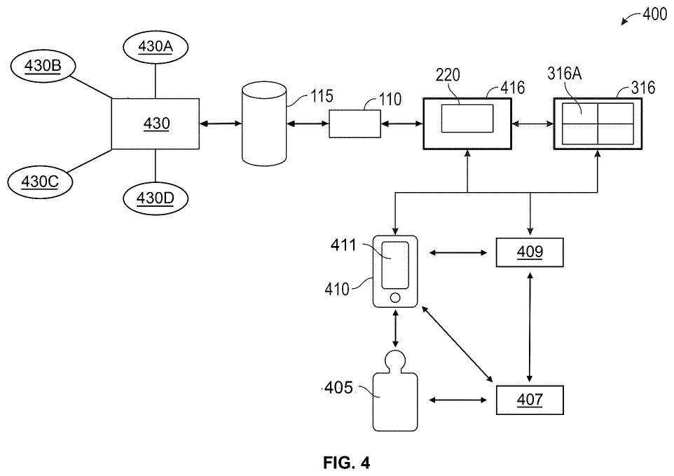

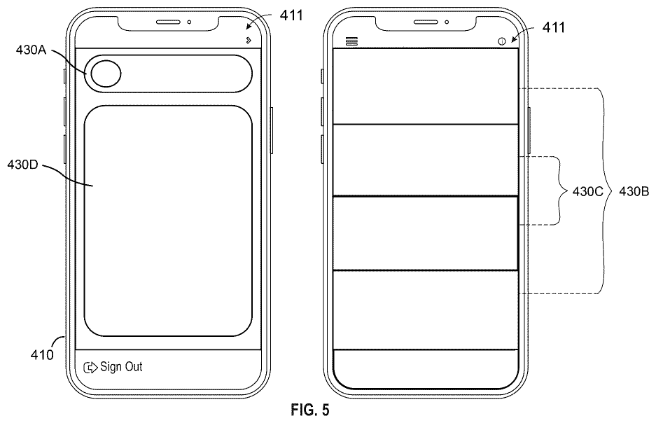

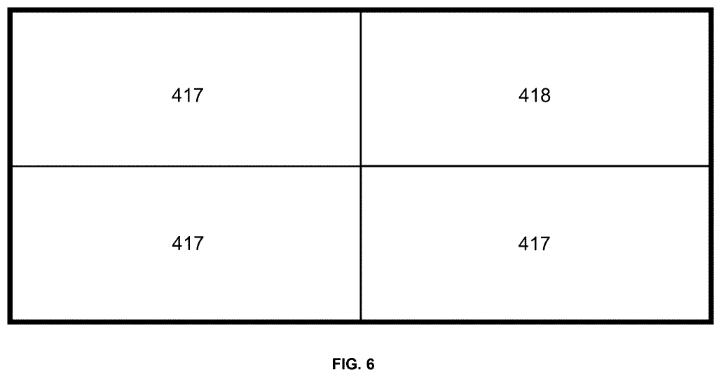

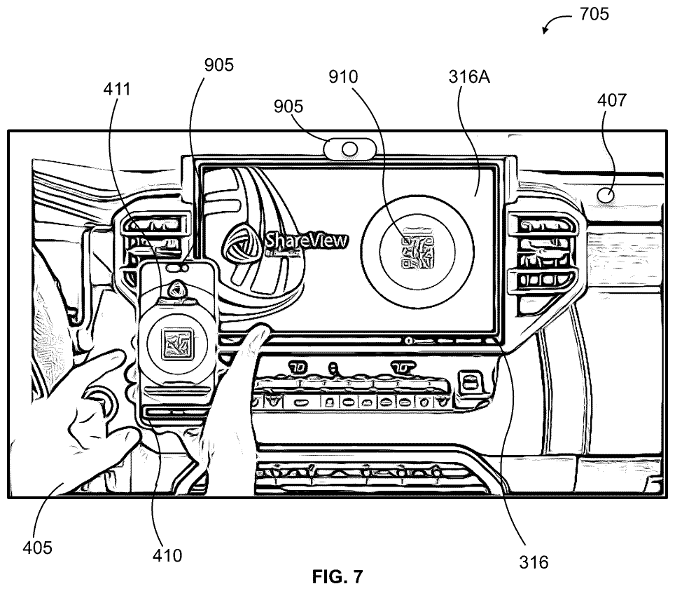

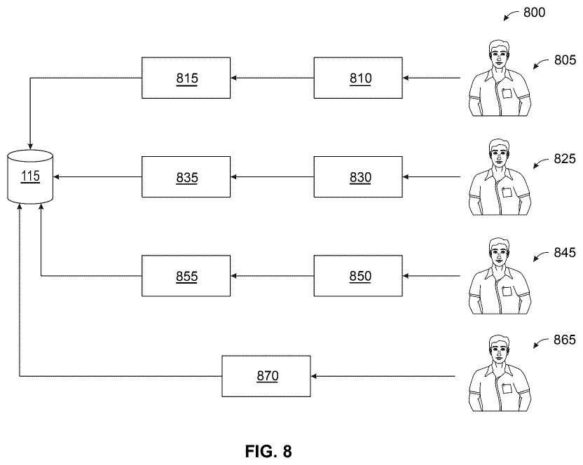

FIGS. 4-8 illustrate embodiments of a system 400 and methods for managing health within an automobile via a display having a plurality of display windows and operably connected to one or more computing devices of a user. FIG. 4 illustrates a preferred embodiment of the system 400 having a computing device 410, display 316, at least one medical device, and a processor operably connected to said computing device, display, and at least one medical device, and non-transitory medium coupled to said processor and containing instructions thereon. FIG. 5 illustrates an example user interface 411 of the computing device 410, wherein a display 316 operably connected to said computing device 410 may split the display user interface into a plurality of display windows containing user data 430A, image data 430B, application data 430C, and patient data 430D. FIG. 6 illustrate an example display user interface 316A having a plurality of display windows showing user data 430A, image data 430B, application data 430C, and patient data 430D pertaining to health of a user, wherein a control board 409 operably connected to said display 316 may receive a computer readable signal from the computing device 410 containing said user data 430A, image data 430B, application data 430C, and patient data 430D. FIG. 7 illustrates the system 400 being used by a user 405 within an environment 700 to manage health. FIG. 8 illustrates permission levels 800 that may be utilized by the present system 400 for controlling access to user content such as user data 430A, image data 430B, application data 430C, and patient data 430D. It is understood that the various method steps associated with the methods of the present disclosure may be carried out as operations by the system 400 shown in FIGS. 4-7.

The system 400 generally comprises a computing device 410 having a user interface 411, medical device having at least one sensor configured to measure patient data of a user, display operably connected to said computing device and said medical device, processor 220 operably connected to said computing device, medical device, and display, and non-transitory computer-readable medium (CRM) 416 coupled to said processor 220 and having instructions stored thereon. Some preferred embodiments may further comprise a camera 905 operably connected to computing devices 410, displays 316, and/or secondary security devices. In one preferred embodiment, a database 115 may be operably connected to the processor 220 and the various data of the system 400 may be stored therein, including, but not limited to, user data 430A, image data 430B, application data 430C, and patient data 430D. In some preferred embodiments, the displays 316 may further comprise a display user interface 316A having a plurality of display windows configured to present the various data of the system 400 therein, wherein control boards 409 of the displays 316 may be configured to receive the various data of the system and arrange the plurality of display windows within the display user interface. In yet another preferred embodiment, a wireless communication interface may allow the processors 220 of the system 400 to receive and transmit the various data of the system therebetween.

Though some embodiments may mention a single computing device 410 of a user 405, one with skill in the art will recognize that multiple computing devices 410 of multiple users may be used without departing from the inventive subject matter described herein. Additionally, though some embodiments may refer to a single display, one with skill in the art will recognize that multiple displays may be linked together in a way that creates a “single” display that may be used in a manner not departing from the inventive subject matter described herein. For instance, four OLED televisions may be linked together in way that creates a multi-display that the system may use as a “single” display. Additionally, one with skill in the art will recognize that a plurality of displays may be controlled by a single control board, and the single control board may manage the plurality of display windows about the display user interfaces of the plurality of displays. In yet another preferred embodiment, two or more control boards of two or more displays may be operably connected to one another and manage the plurality of display windows about the display user interfaces of the plurality of displays in collaboration with one another. Accordingly, one with skill in the art will recognize that displays may be used in combination with one or more control boards and one or more computing devices in a number of ways without departing from the inventive subject matter described herein.

Generally, the system is designed to identify users of the system and monitor health of the users. Users may operably connect to display devices via computing devices and select data to be presented within a display user interface of the display. In a preferred embodiment, a patient user may communicate with a healthcare professional user via the system, wherein the users may manipulate their computing devices to control contents presented on displays in a way that allows the users to interact with one another. For instance, a user in the automobile may use a secondary security means to associate a first computing device of the user with a first display within the automobile. The user may then manipulate the user interface of the computing device in way that connects the first display within the automobile to a second display of a healthcare professional, wherein a second computing device of the healthcare professional is operably connected to the second display. The user in the automobile may choose content to present within the display windows of the display user interface of the first display, and the content chosen by the user within the automobile may also be presented to the healthcare professional via the plurality of display windows of the display user interface of the second display. Alternatively, the healthcare professional may choose content to present within the display windows of the display user interface of the second display, and the content chosen by the healthcare professional may also be presented to the user within the automobile via the plurality of display windows of the display user interface of the first display. Content chosen by the users to be displayed within the plurality of display windows preferably comprises image data.

In a preferred embodiment, a control board 409 of a display 316 receives user data 430A, image data 430B, application data 430C, and/or patient data 430D from a computing entity 200 and/or medical device. The control board 409 may then present said user data 430A, image data 430B, application data 430C, and/or patient data 430D via the display 316 in the display user interface 316A. In another preferred embodiment, the display may be configured to receive user data 430A, image data 430B, application data 430C, and/or patient data 430D via a server and/or database when selected by a user via the user interface of the computing device and/or the display user interface of the display. In a preferred embodiment, the user data 430A, image data 430B, application data 430C, and/or patient data 430D is streamed/mirrored from the computing entity 200, server, and/or database to the control board 409, wherein the control board 409 inserts said streamed/mirrored image data 430D into the display user interface 316A. Alternatively, the control board 409 may manipulate the image data 430D and/or display user interface 316A based on commands received from an input device. In one preferred embodiment, the display user interface 316A may also comprise a control window, which may allow a user 405 to control the layout of the display user interface 316A. For instance, a user 405 may choose a layout that separates the display user interface 316A into multiple windows arranged in a particular way. In some embodiments, the control window may allow a user to alter the size and orientation of a display window of the display user interface. Alternatively, an input device having a plurality of layouts thereon may be used to manipulate the layout of the display user interface 316A. The input device may be connected to the system 400 via a wired or wireless connection. In a preferred embodiment, the input device transmits a computer readable signal containing instructions to the control board 409, which the control board 409 uses to manipulate data presented via the display user interface 316A.

In a preferred embodiment, a user 405 logs into a user profile of the system before accessing the various features of a display, allowing the system to verify the identity of the user. A user interface 411 of a computing device 410 allows a user to input login credentials and/or commands. A processor 220 operably connected to said computing device and said display 316 sends the login credentials and/or commands to a control board of said display via a computer readable signal, wherein said login credentials and/or commands of said computer readable signal allow access to said display should they be associated with a user profile having sufficient permission levels. A user may then manipulate the user interface of the computing device in a way that allows said user to choose various data of the system to be presented on the display for review. In some preferred embodiments, a user 405 may be required to use a secondary security method to access a display to present the various data of the system. For instance, a user 405 may be required to use a camera of their computing device 410 to scan a predefined pattern, such as a bar code or a QR code, that is presented on a display 316, which may associate that user with a particular display.

In a preferred embodiment, displays of the system are configured for remote communication. Preferably, a first user uses a secondary security method to link a first computing device to a first display and second user uses a secondary security method to link a second computing device to a second display. Once connected, the users may select the various data of the system which they would like to be presented within a display window of the displays. For instance, a patient in the automobile may use a secondary security method to associate a display within the automobile with their computing device and user profile. One or more healthcare professionals within a medical facility may be logged into a second display of the system, allowing said one or more healthcare professionals to select various data of the system to be presented within the display windows of the display user interface. The displays are preferably operably connected to one another in a way such that data presented within the display windows of the display is the same. However, though the same data may be presented within the display windows of operably connected displays, the display windows may or may not be organized in the same manner within the display user interfaces of the displays. In a preferred embodiment, each control board of a display controls how the content is organized within display windows of the display user interface.

Medical devices 407 that may be operably connected to the computing device and/or display include, but are not limited to, heart rate monitors, electrocardiograms, pulse oximeters, blood pressure meters, blood glucose meters, smart watches, or any combination thereof. In one preferred embodiment, the medical devices 407 are immovably secured to the display. For instance, the display may comprise a blood pressure meter that cannot be removed by a user 405. A user sitting in the front seating area of the vehicle may position their arm within a cuff of the blood pressure meter such that the user's blood pressure may be measured and saved by the system as patient data 430B. In other preferred embodiments, medical devices 407 are moveably attached to the display. For instance, a pulse oximeter may be moveably secured to the display in a way such that a user 405 may remove said pulse oximeter from a mount of said display so that blood oxygen data may be collected and saved in the form of patient data 430B. In yet another preferred embodiment, medical devices 407 may be configured to operably connect to the display and/or computing device. For instance, a user's smart watch configured to collect patient data 430B may be used by the system to inform a medical professional as to the health of the user 405. For instance, an electrocardiogram positioned about a steering wheel may require that a user 405 touch two electrodes of said electrocardiogram, one with each hand, wherein said electrocardiogram is operably connected to the display in a way such that a user 405 may measure heart activity of the driver while within the vehicle. Accordingly, medical devices may be connected to the display in a plurality of ways without departing from the inventive subject matter as described herein.

In some preferred embodiments, the system 400 may further comprise a secondary security device. Devices that may act as the secondary security device may include, but are not limited to, biometric devices, key cards, wearables, or any combination thereof. In a preferred embodiment, devices that may act as the biometric devices include, but are not limited to, contact biometric devices, such as fingerprint scanners and hand geometry scanners, and/or non-contact biometric devices, such as face scanners, iris scanners, retina scanners, palm vein scanners, and voice identification devices. In some embodiments, the secondary security device may be operably connected to the computing device 410 and/or display 316 in a way such that it is in direct communication with the computing device 410 and/or display 316 and no other computing device 410 and/or display 316. For instance, the secondary security device in the form of a facial recognition camera may be securely and directly connected to a control board 409 of the display 316 such that a user 405 must biometrically scan their face prior to the system allowing access to the various data and features of the system. In some preferred embodiments, biometric data associated with a user is saved in a user profile as user data, which the system uses to verify a user's identity. For instance, secondary security devices may be securely and directly connected to a first computing device and a second computing device in a way such that both a first user of the first computing device and a second user of the second computing device must biometrically scan thumbprints prior to the system allowing the first user and second user to access data of the system.

In a preferred embodiment, key cards and wearables preferably comprise a secure transmitter configured to transmit login credentials to the computing device and/or control board of the display. Wearables having a secure transmitter include clothing and accessories, such as shirts, pants, jackets, belts, shoes, wristbands, watches, glasses, pins, nametags, etc., that have said transmitter attached thereto and/or incorporated therein. The secure transmitter preferably contains login credentials in the form of a unique ID, which may be conveyed to a computing device and/or control board of a display 316 in the form of a computer readable signal. Unique IDs contained within the computer readable signal that has been broadcast by the transmitter may include, but are not limited to, unique identifier codes, social security numbers, personal identification numbers (PINs), etc. For instance, a computer readable signal broadcast by a secondary security device in the form of a wrist band may contain information that will alert the control board of the display 316 that a particular user 405 is within a certain range, which may cause the system 400 to allow a user to access data of the system if additional steps are taken.

Types of devices that may act as the transmitter include, but are not limited, to near field communication (NFC), Bluetooth, infrared (IR), radio-frequency communication (RFC), radio-frequency identification (RFID), and ANT+, or any combination thereof. In an embodiment, transmitters may broadcast signals of more than one type. For instance, a transmitter comprising an IR transmitter and RFID transmitter may broadcast IR signals and RFID signals. Alternatively, a transmitter may broadcast signals of only one type of signal. For instance, identification (ID) cards may be fitted with transmitters that broadcast NFC signals containing unique IDs associated with a particular user, wherein displays equipped with NFC receivers must receive said NFC signals containing unique IDs before access to one or more features of the display user interface may be granted.

Use of secondary security devices may be used solely or in addition to secondary security methods of the system, allowing the system to have flexible multifactor identification. Simultaneous use may be beneficial to prevent unauthorized access to data of the system and/or communications between a user a healthcare professional. For instance, a user may use both a secondary security method and biometric scanner for identification purposes before allowing a user to access the various features of the system. In another preferred embodiment, the system may use a secondary security method for identification purposes and a car key for activating other features of the system. For instance, a user may start a vehicle using a car key having a secure transmitter, which may cause the display to activate. The user may then use a secondary security method to allow the system to identify the user and associate a computing device of the user with the display of the vehicle. Medical devices of the user may transmit patient data to the display and/or computing device where it may be used by the system to perform the various functions described herein.

In a preferred embodiment, the various data of the system 400 may be stored in user profiles 430. In a preferred embodiment, a user profile 430 is related to a particular user 405. A user 405 is preferably associated with a particular user profile 430 based on a username. However, it is understood that a user 405 may be associated with a user profile 430 using a variety of methods without departing from the inventive subject matter herein. Types of data that may be stored within user profiles 430 of the system 400 include, but are not limited to, user data 430A, image data 430B, application data 430C, and patient data 430D. Some preferred embodiments of the system 400 may comprise a database 115 operably connected to the processor 220. The database 115 may be configured to store user data 430A, image data 430B, application data 430C, and patient data 430D within user profiles 430 and/or separately. As used herein, user data 430A may be defined as personal information of a user 405 that helps the system 400 identify the user 405 and their interests. Types of data that may be used by the system 400 as user data 430A includes, but is not limited to, a user's name, username, social security number, phone number, email address, physical address, gender, age, or any combination thereof.

As used herein, image data 430B may be defined as photographic or trace objects that represent the underlying pixel data of an area of an image element, which is created, collected, and stored using image constructor devices, such as a camera. For instance, the system may use image data obtained via a scanning device and/or a secondary security device to confirm the identity of a user. For instance, image data of patient data may be transmitted to the display and presented to the user where it may be reviewed/manipulated by a medical professional to determine the health of the user. For instance, image data of a display application may be transmitted to the display from the computing device, server, and/or database where it may be manipulated by the control board within the plurality of display windows of the display user interface. As used herein, patient data may be defined as information related to a user's medical record, which may usually be found within an electronic health record. Types of data that may be used by the system 400 as patient data includes, but is not limited to, encounter notes, lab/image reports, orders, medications, guidelines, assessments, interventions, pathological reports, or any combination thereof.

Application data may be defined as instructions that cause a display application of the display to perform an action. In one preferred embodiment, the system may determine whether a user application of the computing device is compatible with a display application of the display. If it is determined that the display application and user application are compatible, application data may be transmitted to the display from the computing device in lieu of image data. The display application is controlled by the control board of the display and inserted into a display window of the display user interface. Instructions input into a compatible user application are transmitted to the control board from the computing device and are used by the control board to perform actions of the display application, reducing the amount of data transferred between the computing device and display. For instance, a medical device operably connected to the computing device may transmit patient data to a user application version of a health application of the computing device. A display application version of the health application and the user application version of said health application may be compatible in a way such that a user may open the user application version on their computing device and subsequently instruct the system (via the user interface) to display the user application version in a display window of the display user interface. The processor of the control board may then determine if the display application version of the health application is compatible with the user application version of the health application. If the display application version and user application version are compatible, the control board may open the display application version of the health application locally and manipulate it via instructions received from the computing device as patient data is received or actions are taken via the user application version. If the display application version and user application version are not compatible, the control board may receive image data of the user application version and present it within a display window of the display user interface.

As previously mentioned, some preferred embodiments of the display 316 may further comprise a control board 409. The control board 409 comprises at least one circuit and microchip. In another preferred embodiment, the control board 409 may further comprise a wireless communication interface, which may allow the control board 409 to receive instructions from an input device controlled by a user 405. In a preferred embodiment, the control board 409 may control the plurality of display windows of the display user interface 316A. The microchip of the control board 409 comprises a microprocessor and memory. In another preferred embodiment, the microchip may further comprise a wireless communication interface in the form of an antenna. The microprocessor may be defined as a multipurpose, clock driven, register based, digital-integrated circuit which accepts binary data as input, processes it according to instructions stored in its memory, and provides results as output. In a preferred embodiment, the microprocessor may receive the various data of the system from a server 110 and/or database 115 via the wireless communication interface.

As mentioned previously, the system 400 may comprise a user interface 411. A user interface 411 may be defined as a space where interactions between a user 405 and the system 400 may take place. In an embodiment, the interactions may take place in a way such that a user 405 may control the operations of the system 400. A user interface 411 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, brain-computer interfaces (BCIs), and graphical user interfaces, or any combination thereof. The system 400 may present data of the user interface 411 to the user 405 via a display 316 operably connected to the processor 220. A display 316 may be defined as an output device that communicates data that may include, but is not limited to, visual, auditory, cutaneous, kinesthetic, olfactory, and gustatory, or any combination thereof.

In some preferred embodiments, the user interface and/or display user interface may comprise additional controls that allow users of the system to manipulate how the various data of the system is presented within the display windows. In a preferred embodiment, access to these features is based on permission levels of the user. For instance, the system may be configured in a way such that a user may only choose certain information to be presented within the display windows of the display user interface should they have the appropriate permissions within their user profile. For instance, the system may be configured in a way such that a user may only alter the arrangement of display windows about the display user interface should they have appropriate permissions within their user profile. For instance, the system may be configured in a way such that display windows containing particular data may be presented to a first user having appropriate permissions via a first display but not to a second user of a second display not having appropriate permissions. Accordingly, one with skill in the art will understand that user data 430A, image data 430B, application data 430C, and patient data 430D may be used by the system multiple ways to carry out various functions of the system without departing from the inventive subject matter described herein.

In a preferred embodiment, the control board 409 of the display 316 receives image data from the computing device, server 110, and/or database 115 and may then present said image data 430D via at least one display window of the display user interface 316A of a display 316, as illustrated in FIGS. 4-7. In a preferred embodiment, image data is streamed/mirrored from the computing device, database 115, and/or server 110 to the control board 409, wherein the control board 409 inserts said streamed/mirrored image data 430D into said at least one display window. Alternatively, the control board 409 may automatically select a layout of the display user interface 316A, wherein said layout may be determined based on a plurality of variables, including, but not limited to, number of users, type of content being viewed by the user(s) 405, user preferences, user location, or any combination thereof. For instance, the control board 409 may select a layout of a display user interface 316A comprising a split screen having two display windows configured to present image data of an X-ray in a first display window and image data of an application in a second display window, wherein said application may be configured to present patient data collected from one or more medical devices. For instance, a first control board 409 of a first display may select a layout of a display user interface 316A of the first display comprising four display windows and communication window, wherein a healthcare professional is presented within the communication window, wherein image data containing patient data is presented within the four display windows. A second control board 409 of a second display may select a layout of a display user interface 316A of the second display comprising four display windows and communication window, wherein a patient is presented within the communication window, wherein image data containing patient data is presented within the four display windows. In a preferred embodiment, the control board 409 has one or more of the display windows include information unrelated to health data controlled by a personal computing device 410. For instance, one or more windows might contain navigational information like a map or written directions.

Information presented via a display 316 may be referred to as a soft copy of the information because the information exists electronically and is presented for a temporary period of time. Information stored on the non-transitory computer-readable medium 416 may be referred to as the hard copy of the information. For instance, a display 316 may present a soft copy of visual information via a liquid crystal display (LCD), wherein the hardcopy of the visual information is stored on a local hard drive. For instance, a display 316 may present a soft copy of audio information via a speaker, wherein the hard copy of the audio information is stored in RAM. For instance, a display 316 may present a soft copy of tactile information via a haptic suit, wherein the hard copy of the tactile information is stored within a database 115. Displays 316 may include, but are not limited to, cathode ray tube monitors, LCD monitors, light emitting diode (LED) monitors, gas plasma monitors, screen readers, speech synthesizers, haptic feedback equipment, virtual reality headsets, speakers, and scent generating devices, or any combination thereof.

The database 115 may be operably connected to the processor 220 via wired or wireless connection. In a preferred embodiment, the database 115 is configured to store user data 430A, image data 430B, application data 430C, and patient data 430D within user profiles 430. Alternatively, the user data 430A, image data 430B, application data 430C, and patient data 430D may be stored within user profiles 430 on the non-transitory computer-readable medium 416. The database 115 may be a relational database such that the user data 430A, image data 430B, application data 430C, and patient data 430D associated with each user profile 430 within the plurality of user profiles 430 may be stored, at least in part, in one or more tables. Alternatively, the database 115 may be an object database such that user data 430A, image data 430B, application data 430C, and patient data 430D associated with each user profile 430 of the plurality of user profiles 430 may be stored, at least in part, as objects. In some instances, the database 115 may comprise a relational and/or object database and a server 110 dedicated solely to managing the user data 430A, image data 430B, application data 430C, and patient data 430D in the manners disclosed herein.

In a preferred embodiment, the system 400 may use artificial intelligence (AI) techniques to perform functions of the system. In one preferred embodiment, AI techniques may be used to control the number of display windows presented within the display user interface. In another preferred embodiment, AI techniques may be used to organize the plurality of display windows within the display user interface. In yet another preferred embodiment, AI techniques may be used to evaluate patient data collected by the system to assist medical professionals in the evaluation patients. In yet another preferred embodiment, AI techniques may be used by the system to determine when a user is experiencing a medical emergency or is becoming a driving risk. In yet another preferred embodiment, AI techniques may be used to monitor and alter a user's diet. In yet another preferred embodiment, the AI techniques may be used create grocery lists and order food items on said grocery lists. The term “artificial intelligence” and grammatical equivalents thereof are used herein to mean an intelligence method used by the system 400 to correctly interpret and learn from data of the system 400 or a plurality of systems in order to achieve specific goals and tasks through flexible adaptation. Types of intelligence methods that may be used by the system 400 include, but are not limited to, machine learning, neural network, computer vision, or any combination thereof.

The system 400 preferably uses machine learning techniques to perform the methods disclosed herein, wherein the instructions carried out by the processor 220 for said machine learning techniques are stored on the non-transitory computer-readable medium 416, server 110, and/or database 115. Machine learning techniques that may be used by the system 400 include, but are not limited to, classification algorithms, neural network algorithm, regression algorithms, decision tree algorithms, clustering algorithms, genetic algorithms, supervised learning algorithms, semi-supervised learning algorithms, unsupervised learning algorithms, deep learning algorithms, or other types of algorithms. More specifically, machine learning algorithms can include implementations of one or more of the following algorithms: support vector machine, decision tree, nearest neighbor algorithm, random forest, ridge regression, Lasso algorithm, k-means clustering algorithm, boosting algorithm, spectral clustering algorithm, mean shift clustering algorithm, non-negative matrix factorization algorithm, elastic net algorithm, Bayesian classifier algorithm, RANSAC algorithm, orthogonal matching pursuit algorithm, bootstrap aggregating, temporal difference learning, backpropagation, online machine learning, Q-learning, stochastic gradient descent, least squares regression, logistic regression, ordinary least squares regression (OLSR), linear regression, stepwise regression, multivariate adaptive regression splines (MARS), locally estimated scatterplot smoothing (LOESS) ensemble methods, clustering algorithms, centroid based algorithms, principal component analysis (PCA), singular value decomposition, independent component analysis, k nearest neighbors (kNN), learning vector quantization (LVQ), self-organizing map (SOM), locally weighted learning (LWL), apriori algorithms, eclat algorithms, regularization algorithms, ridge regression, least absolute shrinkage and selection operator (LASSO), elastic net, classification and regression tree (CART), iterative dichotomiser 3 (ID3), C4.5 and C5.0, chi-squared automatic interaction detection (CHAID), decision stump, M5, conditional decision trees, least-angle regression (LARS), naive bayes, gaussian naïve bayes, multinomial naïve bayes, averaged one-dependence estimators (AODE), bayesian belief network (BBN), bayesian network (BN), k-medians, expectation maximisation (EM), hierarchical clustering, perceptron back-propagation, hopfield network, radial basis function network (RBFN), deep boltzmann machine (DBM), deep belief networks (DBN), convolutional neural network (CNN), stacked auto-encoders, principal component regression (PCR), partial least squares regression (PLSR), sammon mapping, multidimensional scaling (MDS), projection pursuit, linear discriminant analysis (LDA), mixture discriminant analysis (MDA), quadratic discriminant analysis (QDA), flexible discriminant analysis (FDA), bootstrapped aggregation (bagging), adaboost, stacked generalization (blending), gradient boosting machines (GBM), gradient boosted regression trees (GBRT), random forest, or even algorithms yet to be invented.

In a preferred embodiment, the system may monitor patient data of a user and processes said data using a machine learning technique to determine potential medical issues a user may be experiencing. For instance, the system may obtain audio data from a user and process it using natural language processing (NLP) to discern what symptoms a user believes they have been experiencing in their own words. The system may then use semi-supervised learning to diagnosis a user with an illness. In some preferred embodiments, the system may use semi-supervised learning to create a treatment protocol for the illness that was previously determined. In one preferred embodiment, the system may continuously monitor patient data of a user who is determined to have an illness and use semi-supervised learning to adjust the treatment protocol of said illness based on changes in said patient data over time. In one preferred embodiment, the system may use machine learning techniques to assist a healthcare professional with diagnosing an illness. For instance, a user having a plurality of symptoms indicative of an autoimmune disorder may use the system to interact with a rheumatologist. The system may obtain patient data from the EHR and analyze said patient data via decision tree, supervised learning in order to help the doctor narrow down which autoimmune disorders are most likely. This information may be provided the rheumatologist via a display window of the display user interface and/or user interface of the computing device of the rheumatologist, which may or may not be viewable to the patient.

In a preferred embodiment, the system may monitor patient data of the user and make health related driving recommendations based on said patient data. For instance, the system may be operably connected to a medical device configured to measure blood sugar of a user. Based on a user's blood sugar as measured by the medical device, the system may use unsupervised learning to make dietary recommendations designed to prevent the blood sugar of the driver from getting too low. In some preferred embodiments, patient data collected and processed by the system may be monitored using a machine learning technique to determine when the user may be experiencing a medical emergency and subsequently call emergency medical services to assist the user. For instance, an electrocardiogram and heart rate monitor operably connected to the display may collect patient data related to the heart of the driver. The system may monitor the patient data and stop the engine of the vehicle should it be determined that the user is experiencing a heart related medical emergency. Additionally, the system may alert emergency medical services of the user's geolocation and that said user is experiencing a heart related medical emergency when the patient data so indicates.

In some preferred embodiments, the system 400 monitors the blood alcohol concentration (BAC) of a user 405 while driving. In a preferred embodiment, this function is applied as a legal penalty to an individual convicted of the offense of driving under the influence of alcohol. In another preferred embodiment, this function is applied to an individual convicted of another alcohol related offense. In embodiments where this function is applied as a legal penalty, it is preferred that the system 400 contact emergency services if the user 405 begins driving with a BAC exceeding the legal maximum or drives without affixing a medical device monitoring BAC. The medical device monitoring BAC may comprise a digital breathalyzer, a wrist device measuring alcohol present in sweat, or another means of assessing alcohol concentration in the user. In yet another preferred embodiment, the system 400 does not apply this function as a legal penalty, but as a safety tool for a user who has consumed alcohol and wishes to know if they are able to drive safely and legally. Continuous monitoring by the system 400 in such an embodiment allows real-time assessments that take into account changes in alcohol absorption and metabolism that may alter a user's capacity to drive.

In a preferred embodiment, the system 400 may use more than one machine learning technique to monitor patient data of a user. For instance, the system 400 comprising a microphone may use a combination of NLP and reinforcement learning to discern additional symptoms, such as congestion, hoarseness, and shortness of breath, in addition to symptoms described by said patient. If the system 400 determines that a user 405 has additional symptoms not described by the patient, the system 400 may add those symptoms to a list of symptoms before determining an illness of the user. In another preferred embodiment, the system 400 may actively monitor a user's alertness to determine if a user is becoming a distracted or tired driver. For instance, the system 400 comprising a camera may use a combination of facial emotion recognition (FER) and deep learning to discern alertness of a user 405 while the automobile is in motion. Should the system determine that a driver is below an alertness threshold, the system may recommend that the driver stop the vehicle and rest. In another preferred embodiment, should the system determine that a driver is below an alertness threshold, the system may activate autopilot capabilities of the automobile. In yet another preferred embodiment, the system 400 comprising a camera may use a combination of NLP, FER, and deep learning to identify the onset of an epileptic seizure. Should the system determine that the driver is undergoing an epileptic seizure, the system may activate autopilot capabilities of the vehicle.

In a preferred embodiment, the machine learning techniques comprise instructions configured to create a trained machine learning techniques from at least some training data and according to an implementation of the machine learning techniques, wherein the training data serves as a baseline dataset that may act as the foundational data of the machine learning techniques. The instructions of the machine learning techniques dictate how the machine learning techniques gain knowledge from the various data sources of the system and may comprise various types of programmable instructions that include, but are not limited to, local commands, remote commands, executable files, protocol commands, selected commands, or any combination thereof. The instructions of the machine learning techniques may vary widely, depending on a desired implementation. In a preferred embodiment, instructions may include streamed-lined instructions that instruct the machine learning techniques on how to train the system, possibly in the form of a script (e.g., Python, Ruby, JavaScript, etc.). In another preferred embodiment, the instructions may include data filters or data selection criteria that define requirements for desired results sets created from the various data of the system as well as which machine learning algorithm is to be used.

Training of the machine learning techniques may be supervised, semi-supervised, or unsupervised. In some preferred embodiments, the machine learning systems may use NLP to analyze data (e.g., audio data, text data, etc.) that may be used to train the machine learning techniques. For instance, the system may use natural language processing and deep learning to ascertain a baseline voice of a user when alert, which may be used by the system to determine when a user may be feeling tired. Training of the machine learning techniques may result in baseline machine learning techniques that may serve as AI techniques for performing the various functions of the system in the manners described herein. Baseline machine learning techniques may further be configured to act as passive models or active models. A passive model may be described as a final, completed machine learning model that uses only the baseline data set to establish behavior of the baseline machine learning technique. An active model may be described as a plasticity machine learning model that is dynamic in that it may be updated using both the baseline dataset and data outside of the baseline data set.

In a preferred embodiment, the system may use a passive model to allow for a high degree of control as to how the system manages user interfaces and display windows in the manners described herein. For instance, a passive model may be configured via a private dataset to provide each user of the system with the same driving recommendations. These recommendations may be made by the system regardless of user data that may indicate that particular users have historically preferred certain actions in certain circumstances. A passive model may be especially useful for users having user profiles with little user data from which the machine learning techniques may learn from. In some preferred embodiments, the system may be configured to begin as passive models until a threshold amount of user data has been acquired. Once the threshold amount of user data has been acquired, the system may cause the machine learning techniques to switch to active models, allowing the system to make recommendations to a user that better parallel historical preferences of the user. For instance, a system may be configured to make driving recommendations to the user based on a passive model for the first 30 times a particular category of driving recommendation is made, wherein the system may also be configured to determine driving actions taken by a user after driving recommendations are made by the system. After the system has made 30 driving recommendations in a particular category of driving recommendation, the machine learning techniques of the system may switch to an active machine model for that particular user and make driving recommendations based on the driving actions taken by the user after recommendations had been made by the system.

In some embodiments, an active machine model may be updated in real-time, daily, weekly, bimonthly, monthly, quarterly, or annually using the various data (e.g., to update model instructions, shifts in time, new/corrected private data sets, user data, patient data, etc.), of the system. In some preferred embodiments, the passive machine model may also be updated as new/updated private data sets become available. In a preferred embodiment, machine learning techniques comprise metadata that describe the state of the passive/active model with respect to its updates. The metadata may include attributes describing one or more of the following: a version number, date updated, amount of new data used for the update, shifts in model parameters, convergence requirements, or other information. Because each user of the system may potentially have a unique machine learning technique associated with their user profile due to the personal nature of user data associated with each user profile, such information allows for identifying distinct passive/active models within the system that may be separately managed.

To prevent un-authorized users from accessing other user's information, the system 400 may employ a security method. As illustrated in FIG. 8, the security method of the system 400 may comprise a plurality of permission levels 800 that may grant users 405 access to user content 815, 835, 855 within the database while simultaneously denying users 405 without appropriate permission levels 800 the ability to view user content 815, 835, 855. To access the user content 815, 835, 855 stored within the database 115, users 405 may be required to make a request via a user interface 411. Access to the data within the database 115 may be granted or denied by the processor 220 based on verification of a requesting user's 805, 825, 845 permission level 800. If the requesting user's 805, 825, 845 permission level 800 is sufficient, the processor 220 may provide the requesting user 805, 825, 845 access to user content 815, 835, 855 stored within the database. Conversely, if the requesting user's 805, 825, 845 permission level 800 is insufficient, the processor 220 may deny the requesting user 805, 825, 845 access to user content 815, 835, 855 stored within the database. In an embodiment, permission levels 800 may be based on user roles 810, 830, 850 and administrator roles 870, as illustrated in FIG. 8. User roles 810, 830, 850 allow requesting users 805, 825, 845 to access user content 815, 835, 855 that a user 405 has uploaded and/or otherwise obtained through use of the system 400. Administrator roles 870 allow administrators 865 to access system 400 wide data.

In an embodiment, user roles 810, 830, 850 may be assigned to a user 405 in a way such that a requesting user 805, 825, 845 may view user profiles 430 containing user data 430A, image data 430B, application data 430C, and patient data 430D via a user interface 411. To access the data within the database 115, a user 405 may make a user request via the user interface 411 to the processor 220. In an embodiment, the processor 220 may grant or deny the request based on the permission level 800 associated with the requesting user 805, 825, 845. Only users 405 having appropriate user roles 810, 830, 850 or administrator roles 870 may access the data within the user profiles 430. For instance, as illustrated in FIG. 8, requesting user 1 805 has permission to view user 1 content 815 and user 2 content 835 whereas requesting user 2 825 only has permission to view user 2 content 835. Alternatively, user content 815, 835, 855 may be restricted in a way such that a user may only view a limited amount of user content 815, 835, 855. For instance, requesting user 3 845 may be granted a permission level 800 that only allows them to view user 3 content 855 related to their specific interest but not user 3 content 855 related to the identity of said user 405. In the example illustrated in FIG. 8, an administrator 865 may bestow a new permission level 800 on users 405 so that it may grant them greater permissions or lesser permissions. For instance, an administrator 865 may bestow a greater permission level 800 on other users 405 so that they may view user 3's content 855 and/or any other user's content 815, 835, 855. Therefore, the permission levels 800 of the system 400 may be assigned to users 405 in various ways without departing from the inventive subject matter described herein.

In some preferred embodiments, the system 400 is installed and used in vehicles other than civilian, noncommercial vehicles. Emergency response vehicles like ambulances, fire trucks, and police cars are disproportionately likely to have drivers and passengers subject to stress, fatigue, and injury. In a preferred embodiment, the system 400 is installed in an ambulance or similar medical vehicle and comprises at least one display in the patient bay and at least one display in the driver's compartment. The system 400 in an ambulance would be capable of monitoring the health status of one or more patients in transit, sharing their data in real time with both the driver and the health professional attending to the patient. In another preferred embodiment, the system 400 is linked to a display 316 present at the destination of the ambulance ride so that the healthcare professionals receiving the patient may stay appraised and prepared for the situation. In yet another preferred embodiment, the healthcare professionals at the ambulance's destination or another location can from their personal computing devices communicate with one more displays 316 in the ambulance to communicate advice or instructions and ask for updates. In still another embodiment, the system 400 monitors the ambulance driver or healthcare professional attending the patient for signs of inattentiveness or fatigue as described herein. Importantly, the multiplicity of medical devices present in a typical ambulance are capable of providing a considerable amount of medical information for the training of AI. In a preferred embodiment, this medical data is used to train artificial intelligence programs in other settings.

The subject matter described herein may be embodied in systems, apparatuses, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one peripheral device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, devices, and arrangements of the parts and method stages which have been described and illustrated in order to explain the nature of this inventive subject matter can be made without departing from the principles and scope of the inventive subject matter.

What is claimed is:

1. A system for managing health in an automobile, comprising:
a control board configured to manipulate a plurality of display windows of a display user interface;
a computing device having a user interface and operably connected to said control board,
wherein said computing device assists said control board;
a display operably connected to said computing device and said control board,
wherein said display is secured within a passenger area of a vehicle,
wherein said display is configured to present said display user interface having said plurality of display windows,
wherein said control board manipulates said plurality of display windows of said display user interface presented on said display;
a medical device operably connected to at least one of said computing device or said control board,
wherein said medical device is configured to measure medical data of a user within said vehicle;
wherein said medical device transmits said medical data to said control board,
a processor operably connected to said control board, computing device, and medical device; and
a non-transitory computer-readable medium coupled to said processor,
wherein said non-transitory computer-readable medium contains instructions stored thereon, which, when executed by said processor, cause said processor to perform operations comprising:
determining an identity of said user associated with said medical device within said vehicle,
retrieving a user profile having user data that pertains to said identity,
receiving said medical data from said medical device;
determining, via a machine learning technique, a health management directive based on said medical data and said user data;
adjusting, via said machine learning technique, said health management directive based on said medical data;
presenting, via said display, said health management directive within said plurality of display windows;
presenting, via said display, said medical data within said plurality of display windows;
organizing, via said control board, said plurality of display windows based on adjustments to said health management directive.

2. The system of claim 1, further comprising additional instructions, which, when executed by said processor, cause said processor to perform additional operations comprising:
determining a medical emergency based on said medical data; and
alerting emergency services of said medical emergency;
providing said emergency services with said vehicle's geolocation; and
providing said emergency services with said medical data and said user data relevant to said medical emergency.

3. The system of claim 2, further comprising additional instructions, which, when executed by said processor, cause said processor to perform additional operations comprising:
notifying emergency contacts of said medical emergency,
wherein said emergency contacts are stored in said user profile of said user,
notifying said emergency contacts of an identity of said emergency services notified about said medical emergency.

4. The system of claim 2, further comprising additional instructions, which, when executed by said processor, cause said processor to perform additional operations comprising:
providing, via said display, instructions to said user or other vehicle occupants on immediate steps to take to address said medical emergency.

5. The system of claim 2, further comprising additional instructions, which, when executed by said processor, cause said processor to perform additional operations comprising:
activating vehicle safety features when said medical emergency is determined (like hazard lights or pulling over to a safe location if the vehicle has autonomous capabilities).

6. The system of claim 1, further comprising additional instructions, which, when executed by said processor, cause said processor to perform additional operations comprising:
monitoring said user's alertness based on said medical data; and
generating a driving recommendation when said user's alertness falls below a predetermined threshold.

7. The system of claim 1, further comprising a camera operably connected to at least one of said computing device or said control board,
wherein said camera collects image data used to determine an identity of said user.

8. The system of claim 8, further comprising additional instructions, which, when executed by said processor, cause said processor to perform additional operations comprising:
receiving image data of said user from said camera; and
analyzing, via said machine learning technique, said image data to confirm said identity of said user.

9. A method for managing health in an automobile, comprising steps of:
determining, by a processor, an identity of a user associated with a medical device within a vehicle;
retrieving, by said processor, a user profile having user data that pertains to said identity;
receiving, by said processor, medical data from said medical device;
determining, via a machine learning technique, a health management directive based on said medical data and said user data;
adjusting, via said machine learning technique, said health management directive based on said medical data;
presenting, via a display operably connected to said processor, said health management directive within a plurality of display windows of a display user interface,
wherein said display is within located within a passenger area of a vehicle;
presenting, via said display, said medical data within said plurality of display windows; and
organizing, via a control board operably connected to said processor, said plurality of display windows based on adjustments to said health management directive.

10. The method of claim 9, further comprising:
determining, by the processor, a medical emergency based on said medical data;
alerting, by the processor, emergency services of said medical emergency;
providing, by the processor, said emergency services with said vehicle's geolocation; and
providing, by the processor, said emergency services with said medical data and said user data relevant to said medical emergency.

11. The method of claim 10, further comprising:
notifying, by the processor, emergency contacts of said medical emergency,
wherein said emergency contacts are stored in said user profile of said user; and
notifying, by the processor, said emergency contacts of an identity of said emergency services notified about said medical emergency.

12. The method of claim 10, further comprising:
providing, via said display, instructions to said user or other vehicle occupants on immediate steps to take to address said medical emergency.

13. The method of claim 10, further comprising:
activating, by the processor, vehicle safety features when said medical emergency is determined.

14. The method of claim 9, further comprising:
monitoring, by the processor, said user's alertness based on said medical data; and
generating, by the processor, a driving recommendation when said user's alertness falls below a predetermined threshold.

15. The method of claim 9, further comprising:
receiving, by the processor, image data of said user from a camera operably connected to said processor; and
analyzing, via said machine learning technique, said image data to confirm said identity of said user.

16. A non-transitory computer readable medium containing instructions configured to manage health in an automobile, comprising:
a non-transitory computer-readable medium coupled to a processor,
wherein said non-transitory computer-readable medium contains instructions stored thereon, which, when executed by said processor, cause said processor to perform operations comprising:
determining an identity of said user associated with a medical device within a vehicle;
retrieving a user profile having user data that pertains to said identity;
receiving medical data from said medical device;
determining, via a machine learning technique, a health management directive based on said medical data and said user data;
adjusting, via said machine learning technique, said health management directive based on said medical data;
presenting, via a display located within a passenger are of said vehicle, said health management directive within said plurality of display windows;
presenting, via said display, said medical data within said plurality of display windows; and
organizing, via a control board operably connected to said display and said processor, said plurality of display windows based on adjustments to said health management directive,
monitoring an alertness of said user based on said medical data; and
generating a driving recommendation when said alertness of said user falls below a predetermined threshold.

17. The non-transitory computer-readable of claim 16, further comprising additional instructions, which, when executed by said processor, cause said processor to perform additional operations comprising:
determining a medical emergency based on said medical data;
alerting emergency services of said medical emergency;
providing said emergency services with said vehicle's geolocation; and
providing said emergency services with said medical data and said user data relevant to said medical emergency.

18. The non-transitory computer-readable of claim 17, further comprising additional instructions, which, when executed by said processor, cause said processor to perform additional operations comprising:
notifying emergency contacts of said medical emergency,
wherein said emergency contacts are stored in said user profile of said user,
notifying said emergency contacts of an identity of said emergency services notified about said medical emergency.

19. The non-transitory computer-readable of claim 17, further comprising additional instructions, which, when executed by said processor, cause said processor to perform additional operations comprising:
activating vehicle safety features when said medical emergency is determined (like hazard lights or pulling over to a safe location if the vehicle has autonomous capabilities).

20. The non-transitory computer-readable of claim 16, further comprising additional instructions, which, when executed by said processor, cause said processor to perform additional operations comprising:
receiving image data of said user from a camera operably connected to said processor; and
analyzing, via said machine learning technique, said image data to confirm said identity of said user.
